(19) United States
(12) Patent Application Publication
(10) Pub. No.: US 2010/0160715 A1
(43) Pub. Date: Jun. 24, 2010

(54) METHOD OF MINIMAL INVASIVE TUNNELING

(76) Inventor: Yiming Deng, Mounds View, MN (US)

Correspondence Address: AMS RESEARCH CORPORATION, 10700 BREN ROAD WEST, MINNETONKA, MN, 55343, US

(21) Appl. No.: 12/643,557

(22) Filed: Dec. 21, 2009

Related U.S. Application Data
(60) Provisional application No. 61/140,136, filed on Dec. 23, 2008.

(51) Int. Cl. A61F 2/02

(52) U.S. Cl. 600/30

(57) ABSTRACT

An implantable apparatus and related methods for placement of a sling so as to provide a controllable restriction about a body lumen for restoring controlled fecal continence. The sling is generally inserted through the use of a tunneler for defining tunnels in which the sling may be inserted to provide control of the sphincter. The tunneler can be provided individually or as a set or kit of tunnelers having unique cross-sectional areas such that the size of the tunnels can be increased to accommodated insertion of the sling. The tunneler can include a self-expandable device, which can be adjusted or controlled to create a wider tunnel. The tunneler can include a semi-rigid case or sheet is disposed about the middle part of the sling to protect the mesh, make tissue penetrator easier and allow for adjustment of the mesh following insertion.

### PRIORITY CLAIM

The present application claims priority to U.S. Provisional Application Ser. No. 61/140,136, filed Dec. 23, 2008, and entitled, “METHOD FOR MINIMAL INVASIVE TUNNELING”, which is herein incorporated by reference in its entirety.

### FIELD OF THE INVENTION

The present invention generally relates to an apparatus and method for the treatment of fecal incontinence. More specifically, the present invention involving a method and apparatus for creating a tunnel to position a trans-obturator post-anal sling in a post anal position with minimal dissecting of a patient's tissue.

### BACKGROUND OF THE INVENTION

Even in a healthy person, the voluntary control of gas and stool is a complex process. A circular muscle called the anal sphincter controls the release of stool. In men and women, this muscle wraps around the anal canal, a small tunnel between the rectum and the anus. A healthy sphincter naturally stays tightly closed most of the time. This muscle squeezes the anal canal and prevents stool from leaking out of the bowel. When the sphincter relaxes, the anal canal opens and allows stool to be carried through the anal canal, out the rectum.

Fecal incontinence (FI) is the impaired ability to control stool. Although not a life-threatening disease, symptoms are often distressing and socially incapacitating. For patients who are under nursing care, not only are there sanitary concerns but there exist labor costs associated with disposing of fecal waste after a patient's incontinent episode and cleaning the patient after the event. Other problems include the possible excoriation of the patient's skin when it is exposed to fecal waste for significant amounts of time, and the risk of contamination for patients and nursing personnel from fecal material. Economically, the replacement of soiled bed linens, blankets and gowns compounds the loss of valuable nursing time and effort and increases healthcare costs.

According to published reports, daily or weekly episodes of fecal incontinence occur in approximately 2% of the adult population and in about 7% of healthy, independent adults over the age of 65. Fecal incontinence is second only to dementia as the cause of institutionalization in the elderly and has been variously estimated to affect from about 32% to about 47% of all nursing home residents. In addition, FI accounts for expenses of over $400 million per year for adult diapers alone. The condition arises from a number of causes including spinal bifida, dementia, obstetric injury and side effects of anorectal surgery.

Fecal incontinence is the loss of voluntary control to retain feces in the rectum. In addition to disease and trauma, fecal incontinence can simply be a result of the aging process. Some patients suffering from fecal incontinence may deal with the condition by performing exercises, utilizing biofeedback or managing their diet. For some patients, however, such measures are ineffective. In a healthy human being, the internal and external anal sphincters contract to prevent the escape of waste, the external sphincter being under the voluntary control of the patient. While some patients may exhibit some control over the external sphincter, one or both sphincters can lack sufficient bulk to close the anus and prevent the escape of fecal matter.

One potential method of treating fecal incontinence involves the placement of a sling about the rectum. In the development of a trans-obturator post-anal sling for treatment of fecal incontinence, one challenge is to lay out the mesh (˜2.5×˜5.0 cm) flat at a post-anal position through a 3-5 cm tunnel (between two small incisions).

### SUMMARY OF THE INVENTION

In a representative surgical procedure of the present disclosure, a sling can be inserted to provide control of the sphincter. For a female, placing the sling may involve the following treatment step. First, two small incisions can be made near the groin at the obturator of the pubic bone and one in the vagina. Narrow sling carriers are then passed through the groin incisions and exit through the vaginal incision. Next, a tunneler is applied to facilitate proper placement of the sling. A mesh is then attached to the sling carriers and placed under the rectum. Following placement of the mesh, the sling carriers are removed, the mesh tension is adjusted and the two small incisions are closed. With the present invention, a novel tunneling technology is utilized to facilitate placement of the transobturator sling and reduce the trauma of dissecting.

The obturator foramen is covered by a thick membrane called the obturator membrane. The external and internal obturator muscles cover this membrane. It is a very safe space anatomically, for placement of a sling, i.e., there are no major vascular or nerve structures near the ischiopubic ramus. This is the area that the needle is passed for the sling placement. This disclosure describes methods to dissect tissue inside a needle path using a novel tunneler. The present invention is easy to install, fast, and flexible. It can reduce the trauma of dissection and save time during the surgery and ease recovery.

The present invention relates to one aspect of an implantable apparatus to provide a controllable restriction about a body lumen. One application of the present invention is for restoring control of fecal continence. In applications involving fecal incontinence, placement of the system about the anal canal and proper adjustment can provide substantially normal anatomical function to a patient.

In a first representative embodiment, a tunneler can be made of such materials that have both certain tension and flexibility and with a handle, an enlarged body and a reusable or removable connector. The tunneler can be connected to a needle utilized to penetrate tissue and establish a suitable pathway for placement of a sling. With the guidance or retrieving of the needle, the tunneler can be pulled into the pathway established within the tissue. The tunneler can follow the needle pathway and expand the tunnel with its enlarged body part. The procedure can be sequentially repeated with another tunneler having a larger cross-section to further increase a tunnel diameter if it is necessary. Upon completion of the tunnel, a sling is attached to a tip of the tunneler such that upon retrieving the tunneler, the sling can be placed in the desired position.

In a second representative embodiment, a tunneler can be a self-expandable, football-shaped device, which can be adjusted or controlled through both ends of the tunneler. The tunneler can be inserted into a tissue pathway through a delivery catheter and guided by a needle. When the catheter is withdrawn, the self-expandable, football-shaped device expands wherein the back and forth movement of the self-expandable device along the tunnel results in the diameter of the tunnel increasing.

In a third representative embodiment, a semi-rigid case or sheet can be disposed about a middle part of a sling. The semi-rigid case or sheet provides a number of benefits to the sling including protecting a middle section of a mesh, allowing for easier penetration of tissue and providing a more convenient adjustment mechanism for orienting the mesh after insertion as the semi-rigid case or sheet can be felt from outside the body. Following successful placement of the mesh, the semi-rigid case or sheet can be peeled off, unlocked from the end(s) or otherwise removed from the sling without changing the position of the sling. The semi-rigid case or sheet can provide for one-way or two-way pulling through the tunnel depending on the design.

These and other features and advantages of the present invention will become apparent from the following more detailed description, when taken in conjunction with the accompanying drawings which illustrate, by way of example, the principles of the invention.

### BRIEF DESCRIPTION OF THE DRAWINGS

The invention can be more completely understood in consideration of the following detailed description of various embodiments of the invention in connection with the accompanying drawings, in which:

FIG. 1 is a perspective view of a first embodiment of a tunneler.

FIG. 2 is a cross sectional view of the tunneler body of the first embodiment.

FIG. 3 is a cross sectional view of the tunneler body of an alternate embodiment.

FIG. 4 is a cross sectional view of a set of tunnelers of the first embodiment.

FIG. 5 is a sectional view of the second embodiment of the present invention with the delivery catheter.

FIG. 6 is a sectional view of the present invention expanded outside the delivery catheter.

FIG. 7 is a perspective view of the cross points of the expanding strips.

FIG. 8 is a planar view of the third embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE DRAWINGS

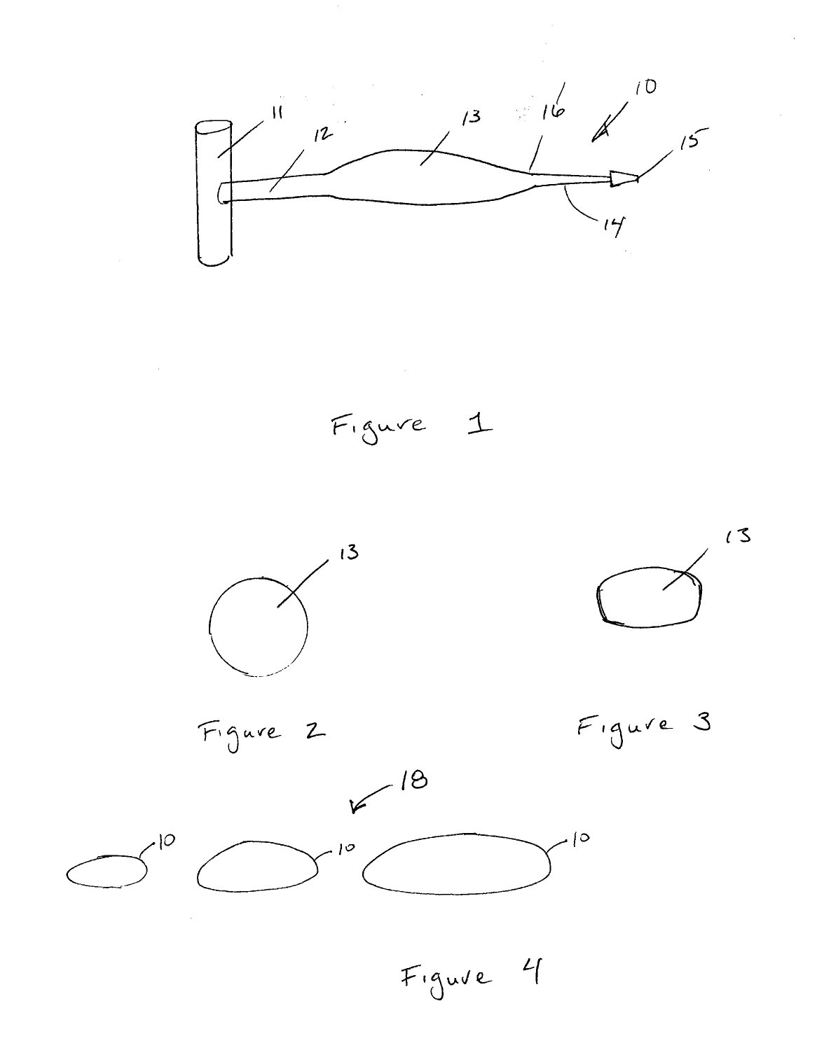

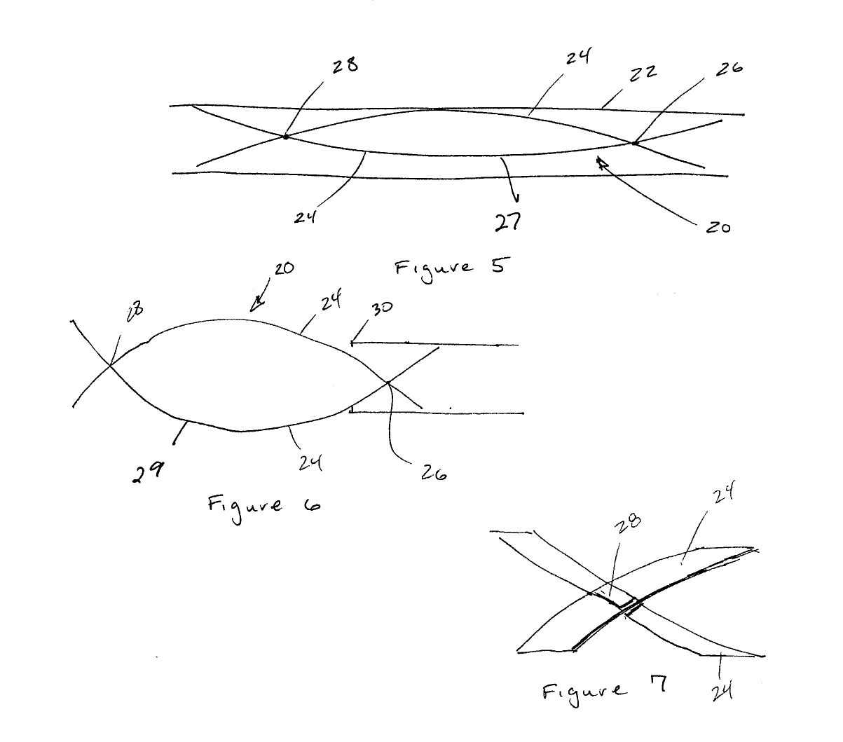

As illustrated in FIG. 1, a representative embodiment of a tunneler 10 according to the present disclosure can comprise a handle 11, a shaft 12, a tunneler body 13, a connector shaft 14 and a connector 15. The handle 11 can be sized as appropriate to allow for control of the tunneler 10. As illustrated, the handle 11 has a diameter sufficient to connect to shaft 12 which extends distally towards tunneler body 13. The tunneler body 13 generally has a wider diameter than shaft 12. Tunneler body 13 can formed so as to have any of a variety of suitable cross-sectional areas. Referring to FIG. 2, an embodiment of tunneler 10 can comprise tunneler body 13 having a substantially circular cross sectional area. In an alternative configuration illustrated in FIG. 3, tunneler body 13 may have a substantially elliptical cross section as illustrated in FIG. 3. Referring again to FIG. 1, a distal end 16 of tunneler body 13 narrows to form connector shaft 14. The length of connector shaft 14 is set so as to conform to and provide for connection of the tunneler 10 to a guide needle (not depicted) that helps draw the tunneler 10 through the tissue. In some representative embodiments, tunneler 10 can comprise a removable connector 15 disposed at the distal end of connector shaft 14. In operation, a plurality of tunnelers 10 can be provided in a tunneler kit 18 or set, wherein each tunneler 10 has a unique diameter and/or cross-sectional area as illustrated in FIG. 4. Tunnel set 18 provides a medical professional the opportunity to gradually increase the size of tunnels formed in tissue through the sequential passing of tunnelers 10, wherein the cross-section of tunneler body 13 for each sequential tunneler 10 is increased in comparison to the previous tunneler 10.

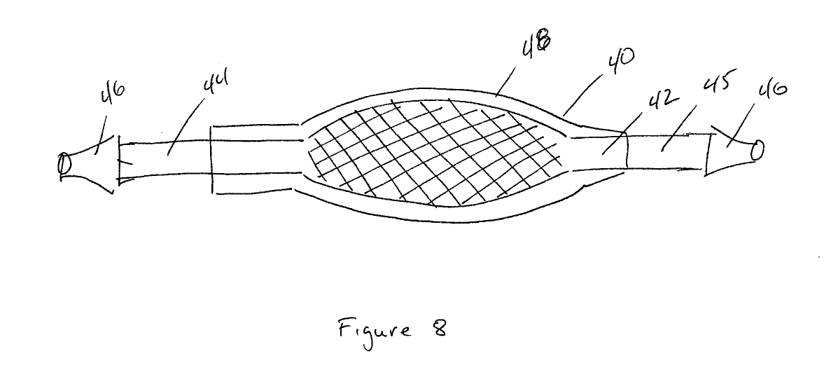

As illustrated in FIGS. 5, 6 and 7, an alternative embodiment of a tunneler 20 is positioned within a delivery catheter 22. The tunneler 20 is generally comprised of a pair of self expanding strips 24 that are connected at cross points 26 and 28. The self expanding strips 24 are held and retained in a compressed state 27 within the delivery catheter 22. Upon the tunneler 20 being expelled and released from a catheter end 30 of the delivery catheter 22 such that tunnel 20 is positioned in the proper tunnel location, strips 24 expand to assume an expanded state 29 illustrated in FIG. 6. Cross points 26 and 28 are defined by a connection for the two strips 24 and provide for attachment of a needle or similar device such that tunneler 20 can be drawn through the tissue. In a representative embodiment, cross points 26 and 28 may involve a slot in one strip or corresponding slots in each expanding strip 24 such that strips 24 can be interlocked at cross points 26 and 28. In operation the tunneler 20 can be selectively expanded according to need by drawing or otherwise directing the cross points 26 and 28 together. In addition, the tunneler 20 may be rotated to the appropriate coordinate to increase tunnel size.

In a third embodiment as illustrated in FIG. 8, an embodiment of a tunneler 40 can be included with a sling 42. Sling 42 can include a mesh sling body 43 disposed between two support arms 44 and 45. Each arm 44 and 45 includes a connector 46 at both ends for operable connection with a needle (not depicted) for drawing/pulling the sling into the tunnel. The tunneler 40 can be a semi-rigid case or sheet that fits around or is woven through the mesh sling body 43. Tunneler 40 extends partially across the respective support arms 44 and 45.

In operation, the sling 42 and tunneler 40 are directed though the appropriate tissue passage by a single needle or dual needle approach. A hard case 48 surrounding the mesh sling body 43 creates the space needed for the sling without damaging the mesh 43. The hard case 48 also assists in the orientation of the mesh 43 as it is a planar structure. The hard case 48 can then be released from the mesh body 43 and removed. In one embodiment, the hard case 48 is peeled off in a single piece or in multiple pieces along preset perforations. It is envisioned that the hard case 48 can include access points providing for wire attachments to the connectors 46.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

1. A tunneler apparatus for insertion into a tissue pathway; comprising:
a tunneler body having a proximal end, a middle portion, and a distal end, the proximal end and the distal end having end diameters smaller than a tissue pathway diameter, and wherein a middle portion diameter is larger than the tissue pathway diameter.

2. The tunneler apparatus of claim 1, wherein the distal end comprises a connector, the connector adapted to attach to a needle.

3. The tunneler apparatus of claim 1, wherein a cross-sectional area of the middle portion is substantially circular.

4. The tunneler apparatus of claim 1, wherein a cross-sectional area of the middle portion is substantially elliptical.

5. The tunneler apparatus of claim 1, further comprising a handle connected to the proximal end of the tunneler body.

6. The tunneler apparatus of claim 1, further comprising a sling, wherein at least a portion of the sling is located within a hard case defining the middle portion.

7. The tunneler apparatus of claim 6, wherein the sling includes a first support arm, a second support arm, and a mesh body, the mesh body disposed between the first support arm and the second support arm.

8. The tunneler apparatus of claim 6, wherein the hard case is removable without substantially changing a position of the sling when implanted in tissue.

9. The tunneler apparatus of claim 1, wherein the first end portion, second end portion, and tunneler body are defined by a pair of self-expanding, arcuate strips, each self-expanding, arcuate strip having a first end and a second end, wherein the first ends and the second ends are operably connected at a first connection point and a second connection point, wherein a compression force applied to the pair of self-expanding, arcuate strips result in the middle portion diameter decreasing.

10. A method of expanding a needle pathway for placement of a support sling, comprising:
providing a tunneler apparatus a proximal end, a middle portion and a distal end, the middle portion having a middle portion diameter larger than a needle pathway diameter;
inserting the distal end into the needle pathway; and
pulling the middle portion through the needle pathway to increase the needle pathway diameter.

11. The method of claim 10, further comprising:
coupling a sling to the tunneler apparatus such that the sling is inserted into the needle pathway substantially simultaneously with the tunneler apparatus.

12. The method of claim 11, further comprising:
removing the tunneler apparatus from the needle pathway while leaving a portion of the sling within the needle pathway.

13. The method of claim 10, further comprising:
removing the tunneler apparatus from the needle pathway; and
inserting a second tunneler apparatus into the needle pathway; the second tunneler apparatus having a second tunneler body with a second middle portion, the second middle portion a second middle portion diameter larger than the middle portion diameter of the original tunneler apparatus.

14. The method of claim 10, wherein the tunneler body is defined by a pair of self-expanding, arcuate strips, each self-expanding, arcuate strip having a first end and a second end, wherein the first ends and the second ends are operably connected at a first connection point and a second connection point, wherein a compression force applied to the pair of self-expanding, arcuate strips result in the middle portion diameter decreasing, and wherein the method further, comprises:
positioning the self-expanding arcuate strips within a delivery catheter; and
removing the self-expanding arcuate strips from the delivery catheter to increase the middle portion diameter.
